(19) United States
(12) Patent Application Publication
(10) Pub. No.: US 2015/0216783 A1
(43) Pub. Date: Aug. 6, 2015

(54) OXIDISED CELLULOSE

(71) Applicant: CRODA INTERNATIONAL PLC, YORKSHIRE (GB)

(72) Inventor: Patrick James Colver, Cheshire (GB)

(73) Assignee: CRODA INTERNATIONAL PLC, YORKSHIRE (GB)

(21) Appl. No.: 14/422,478

(22) PCT Filed: Aug. 8, 2013
(86) PCT No.: PCT/GB2013/052124

§ 371 Date: Feb. 19, 2015

(30) Foreign Application Priority Data
Aug. 20, 2012 (GB) 1214826.8

(51) Int. Cl. A61K 8/73; A61Q 90/00; C08B 15/02

(52) U.S. Cl. CPC A61K 8/731; C08B 15/02; A61Q 90/00; A61K 2800/413; A61K 2800/612

(57) ABSTRACT

A method of preparing oxidised cellulose and intermediate oxidised cellulose, and compositions or dispersions comprising said oxidised cellulose are disclosed. Cellulose particles with a median volume particle diameter (D(v,0.5)s) in the range from 30 μm to 70 μm are reacted in the presence of a nitroxyl radical catalyst, halide co-catalyst, and oxidant, to produce intermediate oxidised cellulose particles. The intermediate oxidised cellulose particles are neutralised, and shear pressure applied to produce oxidised cellulose particles having a median volume particle diameter value (D(v,0.5)p) of less than D(v,0.5)s, a ratio of D(v,0.5)s to D(v,0.5)p from 1.1 to 5.0:1, and an oxidation value in the range from 15% to 30%. The oxidised particles may be formed in to dispersions, with the dispersions particularly used to provide desired skin feel in personal care compositions.

### CROSS REFERENCE TO RELATED APPLICATIONS

This application is the U.S. National Phase application of PCT International Application No. PCT/GB2013/052124, filed Aug. 8, 2013, and claims priority of Great Britain Application No. 1214826.8, filed Aug. 20, 2012, the disclosures of which are incorporated herein by reference in their entireties for all purposes.

### FIELD OF THE INVENTION

The present invention relates to a method for preparing oxidised cellulose, and dispersions thereof, having advantageous properties.

### BACKGROUND OF THE INVENTION

Oxidised cellulose is a water insoluble derivative of cellulose. It can be produced by selective oxidation of cellulose using an oxidising agent (for example, chlorine, hydrogen peroxide, peracetic acid, or chlorine dioxide). Cellulose which has been oxidised may therefore contain carboxylic acid, aldehyde, and ketone groups, in addition to the originally present hydroxyl groups, with variations provided by varying the oxidant and/or reaction conditions. The resulting oxidised cellulose may provide for modification of physical properties, such that it can be used as a thickener, gelling agent, binder, swelling agent, stabiliser, and/or complexing agent.

Oxidation using systems based on, or which generate in situ, NOₓ are known. In particular, selective oxidation of cellulose can be achieved using catalyst (2,2,6,6-Tetramethylpiperidin-1-yl)oxyl (TEMPO). Selective oxidation of both water soluble and water insoluble polysaccharides, including cellulose, has been achieved using TEMPO catalysts.

WO 2006/001387 discloses a process for the production of cellulose nanofibre, a catalyst for oxidation of cellulose, and a method of oxidation of cellulose. The catalyst 4-hydroxy-TEMPO is disclosed as being used to oxidise cellulose.

US 2007/232838 discloses an alternative oxidation catalyst based upon an adamantine structure. This catalyst is described as being useful for oxidation of secondary hydroxyl groups.

The present invention seeks to provide oxidised cellulose and oxidised cellulose dispersions which can both exhibit improved properties of themselves, and when used in an end use applications (such as in personal care formulations). The present invention further seeks to provide a method of making the oxidised cellulose, and of an oxidised cellulose dispersion having said improved properties.

### SUMMARY OF THE INVENTION

According to a first aspect of the present invention, there is provided a method of preparing oxidised cellulose comprising;
(i) reacting cellulose particles, having a median volume particle diameter value (D(v,0.5)s) in the range from 30 μm to 70 μm, in the presence of at least one nitroxyl radical catalyst, at least one halide co-catalyst, and at least one oxidant, wherein the temperature of the reaction is in the range from 1° C. to 30° C., and the pH is in the range from 8 to 12, to produce intermediate oxidised cellulose particles;
(ii) neutralising said intermediate oxidised cellulose particles; and
(iii) applying shear at a pressure in the range from 200 to 1,500 bar to said neutralised oxidised cellulose particles to produce oxidised cellulose particles having a median volume particle diameter value (D(v,0.5)p) of less than D(v,0.5)s, and a ratio of D(v,0.5)s to D(v,0.5)p in the range from 1.1 to 5.0:1, said oxidised cellulose particles having an oxidation value in the range from 15% to 30%.

According to a second aspect of the present invention, there is provided a method of preparing oxidised cellulose comprising;
(i) reacting cellulose particles, having a median volume particle diameter value (D(v,0.5)s) in the range from 30 μm to 70 μm, in the presence of at least one nitroxyl radical catalyst, at least one halide co-catalyst, and at least one oxidant, wherein the temperature of the reaction is in the range from 1° C. to 30° C., and the pH is in the range from 8 to 12;
(ii) to produce intermediate oxidised cellulose particles having a median volume particle diameter value (D(v,0.5)i) in the range from 35 μm to 80 μm, and a ratio of D(v,0.5)i to D(v,0.5)s in the range from 0.8 to 1.65:1;
(iii) neutralising said intermediate oxidised cellulose particles; and
(iv) applying shear at a pressure in the range from 200 to 1,500 bar to said neutralised oxidised cellulose particles to produce oxidised cellulose particles having a median volume particle diameter value (D(v,0.5)p) of less than D(v,0.5)s, and a ratio of D(v,0.5)s to D(v,0.5)p in the range from 1.1 to 5.0:1, said oxidised cellulose particles having an oxidation value in the range from 15% to 30%.

According to a third aspect of the present invention, there is provided a method of preparing intermediate oxidised cellulose comprising;
(i) reacting cellulose particles, having a median volume particle diameter value (D(v,0.5)s) in the range from 30 μm to 70 μm, in the presence of at least one nitroxyl radical catalyst, at least one halide co-catalyst, and at least one oxidant, wherein the temperature of the reaction is in the range from 1° C. to 30° C., and the pH is in the range from 8 to 12;
(ii) to produce intermediate oxidised cellulose particles having an oxidation value in the range from 15% to 30%, a median volume particle diameter value (D(v,0.5)i) in the range from 35 μm to 80 μm, and a ratio of D(v,0.5)i to D(v,0.5)s in the range from 0.8 to 1.65:1.

According to a fourth aspect of the present invention there is provided intermediate oxidised cellulose particles obtainable by the method of the third aspect.

According to a fifth aspect of the present invention there is provided oxidised cellulose particles obtainable by the methods of either the first aspect or the second aspect.

According to a sixth aspect of the present invention there is provided intermediate oxidised cellulose particles having an oxidation value in the range from 15% to 30%, and a median volume particle diameter value (D(v,0.5)i) in the range from 35μm to 80μm.

According to a seventh aspect of the present invention there is provided oxidised cellulose particles having an oxidation value in the range from 15% to 30%, a median volume particle diameter value D(v,0.5)p of less than 40 μm, and an equivalent spherical diameter value corresponding to 90% of the volume of all the particles (D(v,0.9)p) of less than 120 μm.

According to an eighth aspect of the present invention there is provided a composition comprising in the range from 1 wt. % to 16 wt. % oxidised cellulose particles obtainable by the methods of either the first aspect or the second aspect.

According to a ninth aspect of the present invention there is provided a composition comprising in the range from 1 wt. % to 16 wt. % oxidised cellulose particles having an oxidation value in the range from 15% to 30%, and a median volume particle diameter value (D(v,0.5)p) of less than 40 μm.

### DETAILED DESCRIPTION OF THE INVENTION

Surprisingly, it has been found that use of specific cellulose particles starting material, and oxidation of said cellulose under specific conditions, provides oxidised cellulose which may have desired physical properties in both a non-dispersed and dispersed form. In particular, improved physical properties may include for example improved viscosity, elastic modulus, and viscous modulus. Such properties may provide for rheology modification (e.g. thickening), and improved skin feel when used in, for example, personal care formulations.

As used herein, the terms ‘for example,’ ‘for instance,’ ‘such as,’ or ‘including’ are meant to introduce examples that further clarify more general subject matter. Unless otherwise specified, these examples are provided only as an aid for understanding the applications illustrated in the present disclosure, and are not meant to be limiting in any fashion.

It will be understood that use of the term cellulose particles refers to particles of cellulose based raw material which are suitable for oxidation in accordance with any of the aspects of the present invention. Said cellulose based raw material will be understood to comprise organic polysaccharide compounds having the repeating monomer formula (C₆H₁₀O₅)ₙ, with each glucose monomer unit linked via a glycosidic β(1→4) bond to an adjacent monomer.

The cellulose particles used in the present invention may comprise cellulose comprising from 500 to 20,000 monomer units. Preferably, from 1,000 to 15,000 monomer units. More preferably, from 2,000 to 10,000 monomer units.

The cellulose particles may have high whiteness value as it may be desirable that the resulting oxidised cellulose is white rather than another colour when used in formulations. Preferably the whiteness value of the starting material is greater than 70%. More preferably, greater than 75%. Most preferably, greater than 80%. Particularly, greater than 84%.

Whiteness values may be determined by measuring absolute values in a UV-vis spectrometer at 461 nm.

The cellulose used as the starting material may comprise several known types of cellulose. It is generally known in the art that alpha-cellulose (α-cellulose) is the fraction resistant to 17.5% and 9.45% sodium hydroxide solution under test conditions, whilst beta-cellulose (β-cellulose) is the soluble fraction which is re-precipitated on acidification of the solution. Gamma-cellulose (γ-cellulose) is the fraction remaining in solution.

It will be desired that the cellulose particles used as a starting material have a high α-cellulose content. α-cellulose would generally be more insoluble and would provide for the resulting oxidised cellulose having the desired properties. The cellulose particles used as the starting material may preferably have an α-cellulose content greater than 70 wt. %. More preferably, greater than 80 wt. %. Further preferably, greater than 90 wt. %. Most preferably, greater than 98 wt. %.

The carboxyl content of the cellulose particles starting material may be less than 5 mol. %. Preferably, the carboxyl content of said cellulose particles is less than 1 mol. %.

The cellulose particles preferably have a low ash content. Preferably, the ash content of the cellulose particles is lower than 1 wt. %. More preferably, lower than 0.75 wt. %. Most preferably, lower than 0.5 wt. %.

The cellulose particles preferably have a bulk density in the range from 40 g/1 to 220 g/l. More preferably, in the range from 80 g/l to 180 g/l. Most preferably, in the range from 110 g/l to 160 g/l.

The cellulose particles may preferably be in the form of cellulose fibres. Where the cellulose particles are in the form of fibres or any other non-spherical forms, it will be understood that references to size are to normalised spherical diameters of said fibres or non-spherical particles.

In the form of a distribution of particle sizes, the cellulose particles would have a median volume particle diameter value. It will be understood that the median volume particle diameter refers to the equivalent spherical diameter corresponding to the point on the distribution which divides the population exactly into two equal halves. It is the point which corresponds to 50% of the volume of all the particles, read on the cumulative distribution curve relating volume percentage to the diameter of the particles i.e. 50% of the distribution is above this value and 50% is below. This value is referred to as the “D(v,0.5)” value and is determined as described herein.

Additionally, “D(v,0.9)” and “D(v,0.1)” values can also be referred to, and these values would be the equivalent spherical diameter corresponding to 90% or 10% respectively of the volume of all the particles, read on the cumulative distribution curve relating volume percentage to the diameter of the particles, i.e. they are the points where 10% or 90% of the distribution is above this value and 90% or 10% are below the value respectively.

The particle size values, used to determine the D(v,0.5), D(v,0.1), and D(v,0.9) values, are measured by techniques based on dynamic light scattering analysis, with the specific method as described in further detail herein.

It has been found that the median size and size distributions of the cellulose particles starting material are important parameters in obtaining an oxidised cellulose product having the desired properties.

It will be understood that individual particles may typically aggregate and/or agglomerate to form clusters/bundles of particles/fibres, each comprising a plurality of individual particles. Said aggregates and/or agglomerates will herein be referred to as aggregates. This aggregation may be present in the cellulose particles starting material, and the level aggregation may change during processing of the starting material, synthesis of the intermediate oxidised cellulose particles, and/or during processing of the intermediate oxidised cellulose to form oxidised cellulose.

The cellulose particles starting material may have median, 90%, and 10% particle size values denoted by D(v,0.5)s, D(v,0.1)s, and D(v,0.9)s.

The cellulose particles may have a D(v,0.5)s value in the range from 30 μm to 70 μm. Preferably, in the range from 35 μm to 60 μm. More preferably, in the range from 38 μm to 55μm. Further preferably, in the range from 40 μm to 50 μm. Most preferably, in the range from 43 μm to 47 μm.

The cellulose particles may have a D(v,0.9)s value of less than 350 μm. Preferably, less than 300 μm. More preferably, less than 250 μm. Further preferably, less than 200 μm. Particularly preferably, less than 180 μm.

Preferably, the cellulose particles have a D(v,0.9)s value of greater than 90 μm. Preferably, greater than 100 μm. More preferably, greater than 115 μm.

Most preferably, the cellulose particles may have a D(v,0.9)s value in the range from 130 μm to 170 μm.

The cellulose particles may have a D(v,0.1)s value of less than 25 μm. Preferably, less than 20 μm. More preferably, less than 18 μm. Further preferably, less than 16 μm.

The cellulose particles may have a D(v,0.1)s value of greater than 5 μm. Preferably, greater than 8 μm. More preferably, greater than 10 μm.

Most preferably, the cellulose particles may have a D(v,0.1)s in the range from 11 μm to 15 μm.

The ratio of the values of D(v,0.9)s to D(v,0.1)s represents the width of the particle size distribution of the cellulose particles starting material, and therefore how defined the distribution is around the median particle size value. It has been found that using cellulose particles having particular widths of particle size distribution may provide for oxidised cellulose having desired properties.

The ratio of D(v,0.9)s to D(v,0.1)s values for the cellulose particles may be in the range from 30:1 to 6:1. Preferably, in the range from 27:1 to 7:1. More preferably, from 23:1 to 9:1. Most preferably, from 20:1 to 10:1.

The width of the distribution may also be represented by the difference between the D(v,0.9)s and D(v,0.1)s values. The difference in the D(v,0.9)s and D(v,0.1)s values for the cellulose particles may be in the range from 50 to 450. Preferably, in the range from 70 to 400. More preferably, from 90 to 350. Further preferably, from 110 to 300. Most preferably, from 130 to 280.

It has been found that using starting material having particle sizes as described herein, results in production of oxidised cellulose particles which are suitable for subsequent dispersion. Additionally, the oxidised cellulose provided by said selection of starting material particle sizes may provide subsequently formed dispersions which have desired properties, for example they may exhibit good skin feel.

The weight average molecular weight of the cellulose particles starting material may be in the range from 1,000 to 10,000,000. Preferably, in the range from 50,000 to 5,000,000. More preferably, in the range from 100,000 to 2,000,000.

The cellulose particles used in the present invention are preferably comprised of substantially non-modified and/or non-derivatised cellulose. By modification and derivatisation, it will be understood that these terms relate to chemical modification or chemical derivatisation. Preferably, at least 95 wt. % of the cellulose particles are comprised of non-modified and/or non-derivatised cellulose. More preferably, at least 98 wt. %. Most preferably, at least 99 wt. %.

The cellulose particles starting material, may be homogeneous in that it is comprised of only one specific type of cellulose particles, for example all having identical molecular weights.

In an alternative embodiment, the cellulose particles may be heterogeneous in that they comprise a mixture, such as a mixture having different molecular weights.

The starting materials may typically be derived from natural sources (for example, wood pulp cellulose, cotton derived cellulose, or bamboo derived cellulose), and as such the cellulose particles starting material so derived will comprise multiple similar constituents depending on the source of the starting material.

Preferably, the cellulose particles are derived from wood pulp. In particular, cellulose particles starting material derived from hard woods may be preferred. Most preferably, the cellulose particles may be derived from birch.

Suitable cellulose particles are commercially available, for example, from CreaFill Fibers Corp. of Chestertown, Md., USA under the CreaTech trade mark, or from J. Rettenmaier & Söhne Gmbh of Rosenberg, Germany under the Arbocel trade mark.

The nitroxyl radical catalyst may be selected from a di-tertiary alkyl nitroxyl radical.

The term ‘catalyst’ as used herein refers to a compound that facilitates the reaction of interest, in this case oxidation of cellulose, by lowering the rate-limiting free energy of the transition state of the reaction resulting in a larger reaction rate at the same temperature. However, unlike other reagents of the reaction, the catalysts are not generally consumed by the overall reaction itself.

Nitroxyl radical catalysts for use in the present invention may be selected from those known and/or suitable for use in oxidation of cellulose. In particular, suitable catalysts include (2,2,6,6-tetramethylpiperidin-1-yl)oxyl (TEMPO) or derivatives thereof. Further description of suitable TEMPO based catalysts may be found in WO 95/07303 which is incorporated herein by reference.

Particularly preferred examples of suitable nitroxyl radical catalysts may be selected from: 2,2,6,6-tetramethyl-piperidin-1-yloxyl (TEMPO); 4-amino-2,2,6,6-tetramethylpiperidine-1-oxyl; 4-hydroxy-2,2,6,6-tetramethylpiperidine 1-oxyl; 4-oxo-2,2,6,6-tetramethyl-1-piperidinyloxy; 2,2,6,6-tetramethyl-1-piperidinyloxy; and 4-acetamido-2,2,6,6-tetramethylpiperidine 1-oxyl (AA-TEMPO).

4-acetamido-2,2,6,6-tetramethylpiperidine 1-oxyl (AA-TEMPO) may be particularly preferred.

The amount of catalyst used may be in the range from 0.001 wt. % to 0.1 wt. % of the total reaction mixture. Preferably, in the range from 0.008 wt. % to 0.05 wt. %. More preferably, in the range from 0.01 wt. % to 0.02 wt. %.

The halide co-catalyst may be selected from a halide, or halide salt comprising at least one inorganic cation and a halide anion. Preferably, the co-catalyst is a halide salt.

The halide or halide anions may be selected from bromo or iodo. Preferably, the halide is bromo. The inorganic cation may be selected from sodium, potassium, magnesium, or calcium. Preferably, the inorganic cation is sodium. Most preferably, the halide salt is sodium bromide.

The amount of halide or halide salt used may be in the range from 0.01 wt. % to 1.00 wt. % of the total reaction mixture. Preferably, in the range from 0.35 wt. % to 0.55 wt. %.

The oxidant used may be any material capable of converting nitroxyl radical catalyst to its corresponding oxoammonium salt. It will be understood that the nitroxyl radical catalyst selectively oxidises the C6 alcohol on the monomeric anhydroglucose units of the cellulose particles. The nitroxyl radical catalyst is itself oxidised by hypohalite ions (for example Br—O⁻) created from the addition of the oxidant (for example sodium hypochlorite) and the co-catalyst (for example sodium bromide).

Particularly preferred oxidants are those selected from alkali or alkaline-earth metal hypohalite salts. Suitable examples may be selected from sodium hypochlorite, lithium hypochlorite, potassium hypochlorite, or calcium hypochlorite.

Hypobromite salt is also particularly preferred, and may be added in the form of the hypobromite salt itself, such as sodium hypobromite. Alternatively, the hypobromite salt may be formed in situ from the addition of a suitable oxidant, such as sodium hypochlorite, and an alkali or alkaline-earth metal bromide salt.

Additional oxidants may also be used. These additional oxidants may be selected from hydrogen peroxide in combination with a transition metal catalyst such as methyltrioxorhenium (VII); hydrogen peroxide in combination with an enzyme; oxygen in combination with a transition metal catalyst; oxygen in combination with an enzyme; peroxyacids such as peracetic acid and 3-chloroperoxybenzoic acid; alkali or alkaline-earth metal salts of persulphates such as potassium persulphate and sodium persulphate; alkali or alkaline-earth metal salts of peroxymonosulphates such as potassium peroxymonosulphate; chloramines such as 1,3,5-trichloro-1,3,5-triazine-2,4,6(1H,3H,5H)trione, 1,3-dichloro-1,3,5-triazine-2,4,6(1H,3H,5H)trione sodium salt, 1,3-dichloro-5,5-dimethylhydantoin, 1-bromo-3-chloro-5,5-dimethylhydantoin, and 1-chloro-2,5-pyrrolidinedione; and alkali or alkaline-earth metal salts of ferricyanide.

The additional oxidants may be used alone or in combination with an alkali or alkaline-earth metal bromide salt.

The preferred oxidant is sodium hypochlorite or sodium hypobromite formed from the addition of sodium hypochloride or sodium bromide.

The amount of oxidant used may be in the range from 0.5 wt. % to 10 wt. % of the total reaction mixture. Preferably, in the range from 1 wt. % to 6 wt. %.

The oxidant may have an equivalent oxidising power of up to 60 g active chlorine per mole of polysaccharide anhydrosugar unit (“ASU”). When sodium hypochlorite is used as the oxidant, it may be added in an amount of from about 30 mol. % to 60 mol. % based on the moles of polysaccharide ASU.

The resulting oxidised cellulose may have up to about 40 mol. % carboxyl groups per mole of polysaccharide ASU, preferably up to about 35 mol. %. The resulting oxidised cellulose product may have up to about 2 mol. % aldehyde groups per mole of polysaccharide ASU, preferably up to about 1 mol. %. By limiting the amount of oxidant and controlling its rate of addition under defined aqueous conditions the polysaccharide C-6 carboxyl derivatives are selectively prepared. The amount of remaining aldehyde groups is limited due to quick conversion to carboxyl groups after initial formation.

The cellulose particles are oxidised in the presence of a nitroxyl radical catalyst, a halide co-catalyst, and an oxidant. Preferably, the oxidation of the cellulose particles is performed using AA-TEMPO, sodium bromide, and sodium hypochlorite.

The reaction medium may be an aqueous medium, or a homogeneous mixed medium (for example, an ether/water mixture). Preferably, the reaction may be performed in an aqueous medium.

The process may comprise the steps of adding water to a reaction vessel, adding cellulose particles, preferably in the form of fibres, to the vessel, adding halide co-catalyst and nitroxyl radical catalyst to the vessel, cooling the vessel to a desired reaction temperature, and feeding in the oxidant.

The oxidant may be added to the reaction vessel over a suitable time period. Suitably, the oxidant may be added to the reaction vessel over a time period in the range from 1 to 6 hours. Preferably, over 3 to 5 hours. It has been found that feeding of the oxidant over a defined time period allows for the process to be undertaken without pre-adjustment of the pH, and also significantly reduces the possibility of fast exotherm.

The reaction is performed at a pH in the range from 8 to 12. Preferably, the pH is in the range from 9 to 11. More preferably, the pH is in the range from 9.5 to 10.5.

The pH of the reaction may be maintained at the desired level based on the amount of the reactants added. Additionally, a pH adjusting solution may be added to maintain the pH at the desired level. For example, a sodium hydroxide solution may be added to maintain a pH in the preferred range until completion of the reaction.

The oxidation of cellulose particles is performed at a temperature of from 1° C. to 30° C. Preferably, the temperature is in the range from 4° C. to 28° C. More preferably, the temperature is in the range from 8° C. to 22° C. Most preferably, the temperature is in the range from 10° C. to 20° C.

The temperature may be maintained by suitable heating or cooling as necessary. Additionally, the feed of reactants, for example the oxidant, may be adjusted to maintain the desired temperature. The control of the temperature to the desired range is found to improve quality and yield of the intermediate oxidised cellulose. It has been found that the temperature of the process relates to the properties of the end product.

The cellulose particles may be oxidised to a desired level of oxidation. The desired level of oxidation is in the range from 15% to 30%. Preferably, the level of oxidation may be in the range from 17% to 29%. More preferably, in the range from 20% to 28%.

The level of oxidisation of the oxidised cellulose will be understood to refer to the percentage glucose units oxidised to carboxylic acid as measured by titration (i.e. the proportion of moles of carboxyl in the oxidised cellulose in relation to the proportion of moles of starting glucose units in the cellulose particles). It is assumed that all oxidation takes place at the primary alcohol positions as primary alcohol specific oxidation chemistry is employed. Furthermore it is assumed that all oxidation leads to carboxylic acid formation.

The level of oxidation of the cellulose refers to both the intermediate oxidised cellulose as formed. These levels of oxidation would also apply to the oxidised cellulose as it is understood that no further oxidation takes place during processing of the intermediate cellulose when forming said oxidised cellulose.

The level of oxidation may be determined by using an acid titration method, with the specific method as described in further detail herein.

The level of oxidation may be controlled in the reaction by varying the amount of oxidant present relative to the amount of cellulose particles present. The ratio of number of moles of oxidant to number of moles of oxidised cellulose may be in the range from 1.2 to 2.8:1 respectively. Preferably, in the range from 1.5 to 2.5:1. More preferably, in the range from 1.7 to 2.3:1. Most preferably, in the range from 1.9 to 2.1:1.

The pH of the reaction medium may be reduced by addition of an acidic solution once the desired oxidation level of the cellulose is achieved. Preferably, the pH of the reaction medium is reduced to a pH in the range from 2 to 5. The acidic solution may be a hydrogen chloride solution. Reduction of the pH may allow for easier subsequent filtration and/or centrifugation of the intermediate oxidised cellulose.

The resulting intermediate oxidised cellulose may be in the form of a slurry solution comprising in the range from 5 to 8 wt. % intermediate oxidised cellulose. The intermediate oxidised cellulose is in a non-dispersed form.

Once the desired level oxidation of the cellulose has been achieved, the intermediate oxidised cellulose may also be subject to a washing step. Said washing step may comprise filtration, preferably by membrane filtration, and/or centrifugation in order to remove any excess salts or other reactants.

Preferably, the solution may comprise in the range from 10 to 14 wt. % intermediate oxidised cellulose after said washing step.

The subsequently formed dispersions made from the intermediate oxidised cellulose may be sensitive to ionic materials, the presence of which may act to destabilise the dispersion. It may be understood that the presence of ionic materials destabilises the oxidised cellulose so that it is less available to provide effective dispersion stabilisation. For this reason, ionic materials, e.g. acids, bases, and salts including neutral salts, such as organic or inorganic salts, are desirably present only at low concentrations, or are absent.

Preferably, the slurry solution comprises less than 1 wt. % of salts after said washing step. More preferably, the slurry solution comprises less than 0.5 wt. % of salts. Removal of these reactants may aid subsequent thickening and/or dispersion of the oxidised cellulose.

The intermediate oxidised cellulose particles may preferably be in the form of cellulose fibres.

The proportion of intermediate oxidised cellulose in the form of aggregates compared to the proportion of intermediate oxidised cellulose in the form of individual oxidised cellulose particles may be an important parameter in providing desired physical properties. It has also been found that the intermediate oxidised cellulose, when subsequently dispersed, may provide improved properties when used, for example, in personal care formulations. In particular, it has been found that it may be the individual oxidised cellulose particles which provide many of these desired properties.

The proportion of intermediate oxidised cellulose aggregates compared to the proportion of individual particles is preferably in the range from 0.7 to 1.3:1.3 to 0.7. More preferably, in the range from 0.85 to 1.15:1.15 to 0.85. Most preferably, in the range from 0.95 to 1.05:1.05 to 0.95.

The intermediate oxidised cellulose in the non-dispersed form may have median volume particle diameter sizes, 10%, and 90% values denoted by D(v,0.5)i, D(v,0.1)i, and D(v,0.9)i. Said particle sizes values are analogous to those described with reference to the cellulose particles starting material, and are determined as described herein.

The intermediate oxidised cellulose may have a D(v,0.5)i value in the range from 35 μm to 80 μm. Preferably, in the range from 45 μm to 75 μm. More preferably, in the range from 50 μm to 70 μm. Further preferably, in the range from 55 μm to 65 μm. Most preferably, in the range from 59 μm to 62 μm.

The intermediate oxidised cellulose may have a D(v,0.9)i value of less than 350 μm. Preferably, less than 300 μm. More preferably, less than 250 μm. Further preferably, less than 200 μm. Particularly preferably, less than 180 μm.

Preferably, the intermediate oxidised cellulose particles have a D(v,0.9)i value of greater than 90 μm. Preferably, greater than 100 μm. More preferably, greater than 115 μm.

Most preferably, the intermediate oxidised cellulose may have a D(v,0.9)i value in the range from 130 μm to 170 μm.

The intermediate oxidised cellulose may have a D(v,0.1)i value of less than 30 μm. Preferably, less than 27 μm. More preferably, less than 24 μm. Further preferably, less than 22 μm.

The intermediate oxidised cellulose may have a D(v,0.1)i value of greater than 10 μm. Preferably, greater than 14 μm. More preferably, greater than 18 μm.

Most preferably, the intermediate oxidised cellulose may have a D(v,0.1)i value in the range from 19 μm to 22 μm.

The ratio of the values of D(v,0.5)i to D(v,0.5)s represents the change in the median particle size between the cellulose particles starting material and the intermediate oxidised cellulose particles. The ratio of the values of D(v,0.5)i to D(v,0.5)s may be in the range from 0.8 to 1.65:1. Preferably, in the range from 1 to 1.55:1. More preferably, in the range from 1.1 to 1.5:1. Further preferably, in the range from 1.2 to 1.45:1. Most preferably, in the range from 1.25:1 to 1.4:1.

The ratio of the values of D(v,0.9)i to D(v,0.9)s represents the change in the 90% particle sizes between the cellulose particles starting material and the intermediate oxidised cellulose particles. The ratio of the values of D(v,0.9)i to D(v,0.9)s may be in the range from 0.7 to 1.3:1. Preferably, in the range from 0.8 to 1.2:1. More preferably, in the range from 0.85 to 1.15:1. Further preferably, in the range from 0.9 to 1.1:1. Most preferably, in the range from 0.95 to 1.05:1.

It has been found that by selecting the cellulose particles starting material with particle size values as defined above, and using the selected reaction conditions and reactants as defined, the particle size values may be substantially maintained during the oxidation. In addition, the oxidation process may result in the loss of some of the smaller sized particles resulting in an increase in the median volume particle diameter. This is advantageous as the smaller particles are understood to be detrimental to the desired properties of the intermediate oxidised cellulose if subsequently dispersed, any may if present act to reduce viscosity and elastic modulus.

The intermediate oxidised cellulose may be neutralised. Said neutralisation may be undertaken by addition of any suitable neutralising agent, for example sodium hydroxide solution.

Preferably, the neutralised intermediate oxidised cellulose has a pH in the range from 5.5 to 7.5. More preferably, in the range from 6.0 to 7.0. It is found that neutralisation of the non-dispersed intermediate oxidised cellulose may allow for easier subsequent dispersion.

The non-dispersed intermediate oxidised cellulose is optionally neutralised and salts are removed, and may be in the form of a water swellable fibre.

Said intermediate oxidised cellulose may be subjected to shear to form a workable product for use in formulations. The optionally neutralised intermediate oxidised cellulose may be formed in to a dispersion after being subject to shear. Said dispersion may be formed in water.

The intermediate oxidised cellulose particles may subjected to a homogenisation step to impart shear and to form oxidised cellulose. Preferably, the homogenisation step comprises using a high pressure homogeniser. By varying the pressure of the high pressure homogenisation it may be possible to advantageously control the shear imparted to the oxidised cellulose.

The intermediate oxidised cellulose particles may be homogenised at a pressure in the range from 300 bar to 1,500 bar. Preferably, in the range from 600 bar to 1,200 bar. More preferably, in the range from 800 bar to 1,000 bar.

The resulting oxidised cellulose may be in the form of a dispersion or other composition. The dispersion may comprise in the range from 1 wt. % to 12 wt. % oxidised cellulose. Preferably, in the range from 1.5 wt. % to 10 wt. %. Most preferably, in the range from 7 wt. % to 9 wt. %. The dispersion may be diluted for further processing.

The proportion of oxidised cellulose particles in the form of aggregates compared to the proportion in the form of individual particles may be changed as a result of the homogenisation step. In the homogenised form, the proportion of aggregates compared to the proportion of individual oxidised cellulose particles may preferably be in the range from 1:2 to 20. More preferably, in the range from 1:5 to 15. Most preferably, in the range from 1:8 to 12.

The oxidised cellulose may have median volume particle diameter sizes, 10%, and 90% particle size values denoted by D(v,0.5)p, D(v,0.1)p, and D(v,0.9)p. Said particle sizes values are analogous to those described with reference to the cellulose particles starting material, and are determined as described herein.

The oxidised cellulose may have a D(v,0.5)p value of less than 40 μm. Preferably, in the range from 10 μm to 35 μm. More preferably, in the range from 15 μm to 30 82 m. Further preferably, in the range from 18 μm to 25 μm. Most preferably, in the range from 20 μm to 22 μm.

The oxidised cellulose may have a D(v,0.9)p value of less than 120 μm. Preferably, in the range from 30 μm to 100 μm. More preferably, in the range from 35 μm to 90 μm. Further preferably, in the range from 40 μm to 60 μm. Most preferably, in the range from 45 μm to 55 μm.

The oxidised cellulose may have a D(v,0.1)p value of less than 18 μm. Preferably, less than 15 μm. More preferably, less than 12 μm. Further preferably, less than 10 μm.

The oxidised cellulose may have a D(v,0.1)p value of greater than 5 μm. Preferably, greater than 6 μm. More preferably, greater than 7 μm.

Most preferably, the oxidised cellulose may have a D(v,0.1)p value in the range from 8.0 μm to 9.5 μm.

The ratio of the values of D(v,0.5)s to D(v,0.5)p represents the change in the median particle size between the cellulose particles starting material and the oxidised cellulose. The ratio of the values of D(v,0.5)s to D(v,0.5)p may be in the range from 1.1 to 5:1. Preferably, in the range from 1.4 to 4:1. More preferably, in the range from 1.6 to 3:1. Further preferably, in the range from 1.8 to 2.4:1. Most preferably, in the range from 2.0 to 2.2:1.

The ratio of the values of D(v,0.5)i to D(v,0.5)p represents the change in the median particle size between the intermediate oxidised cellulose and oxidised cellulose. The ratio of the values of D(v,0.5)i to D(v,0.5)p may be in the range from 1.5 to 8:1. Preferably, in the range from 2.0 to 6:1. More preferably, in the range from 2.3 to 4:1. Further preferably, in the range from 2.5 to 3.1:1. Most preferably, in the range from 2.7 to 1.9:1.

The ratio of the values of D(v,0.9)s to D(v,0.9)p represents the change in the 90% particle size value between the cellulose particles starting material and the oxidised cellulose. The ratio of the values of D(v,0.9)s to D(v,0.9)p may be in the range from 1.5 to 8:1. Preferably, in the range from 1.9 to 6:1. More preferably, in the range from 2.2 to 5:1. Further preferably, in the range from 2.5 to 3.5:1. Most preferably, in the range from 2.8 to 3.2:1.

The ratio of the values of D(v,0.9)i to D(v,0.9)p represents the change in the 90% particle size between the intermediate oxidised cellulose and the oxidised cellulose. The ratio of the values of D(v,0.9)i to D(v,0.9)p may be in the range from 1.5 to 8:1. Preferably, in the range from 1.9 to 6:1. More preferably, in the range from 2.2 to 5:1. Further preferably, in the range from 2.5 to 3.5:1. Most preferably, in the range from 2.8 to 3.2:1.

Advantageously, the oxidation process and subsequent homogenisation provide for oxidised cellulose particles, which may be in a dispersed form, and which have a significantly lower median volume particle diameter than either the non-homogenised intermediate oxidised cellulose or the cellulose particles starting material. In addition, it can be see that the particle size distribution after dispersion is significantly more defined than either in the non-homogenised intermediate oxidised cellulose or the cellulose particles starting material, as can be seen by the differences between D(v,0.1) and D(v,0.9) values in each case. The more defined particle size distribution, and the obtained median volume particle diameter provide for an oxidised cellulose with desired properties.

The oxidised cellulose may advantageously provide for a composition having high viscosity in an aqueous medium, and gelling ability. The homogenised oxidised cellulose desirably has suitable storage stability and viscosity appropriate to the end use. Such properties will allow for use in, for example, personal care formulations where good skin feel is required.

It will be understood that viscosity values defined below are based on aqueous dispersions comprising 4 wt. % of oxidised cellulose and measured on a Haake Rheostress 600. Methods of determining zero-shear viscosity, shear viscosity, and yield point values are as described in more detail herein.

Zero-shear viscosity will be understood to represent the viscosity at the limit of low shear rate, i.e. the maximum plateau value attained as shear stress or shear rate is reduced, and is effectively the viscosity of the composition whilst at rest.

The zero-shear viscosity of the oxidised cellulose may be in the range from 6,000 to 30,000 Pa·s. Preferably, the zero-shear viscosity is in the range from 8,000 to 25,000 Pa·s. More preferably, the zero-shear viscosity is in the range from 10,000 to 21,500 Pa·s.

In relation to the oxidised cellulose product, it has been found that the listed zero-shear viscosity ranges provide for improved stability. Specifically, oxidised cellulose having good zero-shear viscosity values provides for shelf-stable compositions, and this is advantageous where, for example, a dispersion is made and not immediately used.

Yield point will be understood to represent the amount of shear required to break the oxidised cellulose, i.e. to get from its zero-shear viscosity to the under shear viscosity. A low yield point would therefore suggest that only a small amount of force was needed.

The yield point of the oxidised cellulose, when measured for a 4 wt. % oxidised cellulose dispersion, dispersed at 600 bar, may be greater than 5 Pa. Preferably, the yield point is greater than 15 Pa. More preferably, the yield point is greater than 30 Pa. Further preferably, the yield point is greater than 40 Pa. Most preferably, the yield point is greater than 50 Pa. Preferably, the yield point is less than 80 Pa. More preferably, less than 70 Pa. Most preferably, less than 60 Pa.

Dispersions may be divided by viscosity into milks and lotions, which typically have a low shear viscosity of from about 100 mPa·s up to about 10,000 mPa·s, and creams which typically have a low shear viscosity of more than about 20,000 mPa·s to about 80,000 mPa·s.

For good skin feel, personal care and cosmetic emulsions are usually shear thinning, and the measured low shear viscosity is only a general guide to whether the product is a milk (or lotion) or cream. Additionally, a further guide to the ability of a dispersion to add to the skin feel or improve other properties in end use formulation may be to look at comparisons of low shear viscosity and the rate at which the viscosity falls from the zero-shear level.

The shear viscosity describes a compositions tendency to shear (the deformation of shape at constant volume) when acted upon by opposing forces, and will be understood to be defined as shear stress over shear strain.

The shear viscosity value of the oxidised cellulose, at 5 revolutions per second (rps), and dispersed at 600 bar, and when measured for an aqueous 4 wt. % oxidised cellulose dispersion, may be in the range from 15 Pa·s to 35 Pa·s. Preferably, in the range from 18 Pa·s to 30 Pa·s. More preferably, in the range from 19 Pa·s to 25 Pa·s.

The shear viscosity value of the dispersed oxidised cellulose, at 40 revolutions per second (rps), and dispersed at 600 bar, and when measured for an aqueous 4 wt. % oxidised cellulose dispersion, may be in the range from 2.5 Pa·s to 4.8 Pa·s. Preferably, in the range from 2.7 Pa·s to 4.4 Pa·s. More preferably, in the range from 2.9 Pa·s to 4.1 Pa·s.

The ratio of the shear viscosity value at 5 rps to the value at 40 rps represents the rate at which shear viscosity falls with increase in applied shear force. The ratio of the 5 rps shear viscosity value to 40 rps shear viscosity value, when measured for an aqueous 4 wt. % oxidised cellulose dispersion, may be in the range from 4.0 to 12.0:1. Preferably, in the range from 5.0 to 10.0:1. Most preferably, in the range from 6.0 to 8.0:1.

The oxidised cellulose is therefore preferably shear thinning, in particular when in the form of a dispersion. It will be understood that, by the term shear thinning, is where the viscosity of the dispersion decreases with an increasing rate of shear stress.

The oxidised cellulose may advantageously provide for a composition having desired elastic/storage modulus values and viscous/loss modulus values in an aqueous medium, in particular when in a dispersion.

The elastic/storage modulus (denoted as G′) is the measure of a sample's elastic behaviour, i.e. a measure of the elastic response of a material). The viscous/loss modulus (denoted as G″) is the measure of the viscous response of a material.

It will be understood that elastic/storage modulus values and viscous/loss modulus (both G′ and G″) described herein are based on aqueous oxidised cellulose dispersions comprising 4 wt. % of oxidised cellulose and being dispersed at 600 bar.

The elastic/storage modulus value (G′) of the oxidised cellulose may be in the range from 800 to 2,000 Pa. Preferably, in the range from 950 to 1,800 Pa. More preferably, in the range from 1,100 to 1,600 Pa. Most preferably, in the range from 1,250 to 1,450 Pa.

The viscous/loss modulus value (G″) of the oxidised cellulose may be in the range from 50 to 300 Pa. Preferably, in the range from 80 to 260 Pa. More preferably, in the range from 120 to 240 Pa. Most preferably, in the range from 170 to 220 Pa.

The ratio of the elastic/storage modulus value (G′) to viscous/loss modulus value (G″) may be related to the viscosity and rheology properties exhibited by the dispersion. In particular, it is desired that there may be a larger difference between the G′ and G″ values as this is understood to provide for better rheology in, for example, personal care formulations resulting in better “skin feel”. The ratio of the G′ value to G″ value, when measured for a 4 wt. % oxidised cellulose, may be in the range from 3.0 to 12.0:1. Preferably, in the range from 4.5 to 10:1. Most preferably, in the range from 6.5 to 8.0:1.

Elastic/storage modulus (G′) and viscous/loss modulus (G″) values are determined using a Haake Rheostress 600 as described in more detail herein.

The dispersion may be formed in to an emulsion using any suitable technique. In particular, the emulsion may be formed using chemical means or physical means. Suitable emulsions may be achieved by subjecting the dispersion to ultrasound and/or by addition of an emulsifier. Preferably, said emulsion is formed by addition of an emulsifier.

Any emulsifier which is suitable may be used. In particular, emulsifiers which are derived partly or entirely from biological, particularly vegetable, source materials may be desired. This possibility may be particularly attractive to formulators of personal care products comprising the oxidised cellulose dispersion.

Suitable emulsifiers may be selected, by way of example, from a fatty acid ester, ether, hemi-acetal or acetal of a polyhydroxylic compound, or a fatty acid amide which is N-substituted with the residue of a polyhydroxylic compound, especially a saccharide fatty acid ester, and a polysaccharide stabiliser. Sugar (saccharide) esters can be used with advantage in this invention as they may provide stable emulsions which can entirely avoid using products manufactured using alkylene oxides, and can allow for dispersions which are derived entirely from “natural” biological sources, particularly vegetable source materials.

The emulsion may comprise emulsifier in an amount of from about 0.02 wt. % to about 5 wt. % by weight of the total emulsion composition. Preferably, from about 0.025 wt. % to 2 wt. % by weight. Most preferably, from about 0.025 wt. % to 1.5 wt. %.

Many other components may be included in the dispersion composition in order to make it suitable for a desired end use formulation (e.g. personal care or cosmetic formulations). These other components may be oil soluble, water soluble, or non-soluble.

Examples of such materials include preservatives such as those based on parabens (alkyl esters of 4-hydroxybenzoic acid), phenoxyethanol, substituted ureas and hydantoin derivatives, perfumes, humectants or solvents such as alcohols, polyols such as glycerol and polyethylene glycols, sunfilter or sunscreen materials including chemical sunscreens and physical sunscreens including those based on titanium dioxide or zinc oxide, alpha hydroxy acids such as glycolic, citric, lactic, malic, tartaric acids and their esters, self-tanning agents such as dihydroxyacetone, antimicrobial, particularly anti-acne components such as salicylic acid, vitamins and their precursors, skin care agents such as ceramides either as natural materials or functional mimics of natural ceramides, phospholipids, vesicle-containing formulations, germanium-containing compounds, botanical extracts with beneficial skin care properties, skin whiteners such as hydroquinone, kojic acid, arbutin and similar materials, skin repair compounds actives such as allantoin and similar series, caffeine and similar compounds, cooling additives such as menthol or camphor, insect repellents such as N,N-diethyl-3-methylbenzamide (DEET) and citrus or eucalyptus oils, essential oils, and pigments, including microfine pigments, particularly oxides and silicates, and ceramic materials such as boron nitride, or other solid components.

The oxidised cellulose dispersion of the present invention is suitable for use a thickener, viscosifier, or stabiliser for emulsions and the like, and as a starting material for further functionalisation.

It has been found that a dispersion in accordance with the present invention is able to hold and achieve required flow properties (rheology), suspend added particles, and be resistant to separation by sedimentation in the form of the dispersion itself, or when formulated as part of an end-use formulation.

The dispersed oxidised cellulose, which may be combined with other organic/inorganic components, give novel hierarchical structures which have functional properties useful in water based formulations (including rheology modifiers). In particular, the oxidised cellulose dispersion may be used as a rheology modifier in home and personal care formulations. The dispersion of the present invention may be used as a thickener, and may replace known synthetic thickeners.

Without wishing to be unduly bound by theory, it has been found that the benefits of the invention may be conferred due to controlling the particle sizes from the starting material through to the dispersed oxidised cellulose particles, oxidation level, temperature, and other conditions for the oxidation reaction. In particular, the resulting benefits include oxidised cellulose that may have good sensory characteristics, and good rheology in that it has high zero shear viscosity but is shear thinning.

All of the features described herein may be combined with any of the above aspects, in any combination.

In order that the present invention may be more readily understood, reference will now be made, by way of example, to the following description.

It will be understood that all tests and physical properties listed have been determined at atmospheric pressure and room temperature (i.e. 25° C.), unless otherwise stated herein, or unless otherwise stated in any referenced test methods and procedures.

Test methods for determining values are as follows:
Particle size values, used to determine the D(v,0.5), D(v,0.1), and D(v,0.9) values, were determined by dynamic light scattering analysis by using a Malvern Mastersizer 2000 with a Hydro 2000SM attachment running on water set at 2,100 rpm. The refractive index of the material is set as 1.53 with an absorbance of 0.1. 12,000 snaps were taken over 12 seconds to obtain the data. An average of three runs was used to determine a final particle size. From the particle size values obtained, D(v,0.5), D(v,0.1), and D(v,0.9) values were readily determined. All particle sizes, unless otherwise stated, are reported in
Level of oxidation was determined by using an acid titration method as outlined in the US Pharmacopia (USP, 1995). The method includes the steps of:
   a) stirring the oxidised cellulose in 1M HCl for 24 hours to remove all excess sodium,
   b) filter washing the oxidised cellulose in distilled water until the wash water has a neutral pH,
   c) drying the oxidised cellulose to remove all moisture,
   d) adding 0.5 g of the dried oxidised cellulose to 50 ml of 2% calcium acetate(aq.), and
   e) titrating against standardised sodium hydroxide.
The number of moles of sodium hydroxide used in the titration will therefore equal the number of moles of carboxyl groups in the oxidised cellulose, and the percentage oxidation level can be calculated from this.
Zero-shear viscosity data was measured on a Haake Rheostress 600 at 25° C. using a 2° 35 mm titanium cone against a plate with a 104 nm gap one day after preparation of the dispersion at 600 bar. Samples were allowed to equilibrate at temperature for 120 seconds before measurements were taken. The yield point and zero-shear viscosities were measured by increasing the shear stress from 0 to 150 Pa.
Shear viscosity was determined by the same method as described for zero-shear viscosity, and slowly ramping the shear rate up to 500 revolutions per second (rps) then slowly ramping down the shear rate back to 0 rps.
Elastic modulus (G′) and viscous modulus (G″) were measured on a Haake Rheostress 600 using 2° titanium cone and plate, oscillating at 10 Hz ramping the shear stress from 0 to 300 Pa (the basic method being the same as the method for determining zero-shear viscosity, except using these parameters).

### EXAMPLE 1

### Preparing Oxidised Cellulose

Water (200 g) was charged into the reaction vessel. The raw material cellulose particles (20 g) were then added slowly to prevent clumps forming. The raw material cellulose particles had a D(v,0.5)s value of 45.067, a D(v,0.1)s value of 12.68, and a D(v,0.9)s value of 152.139.

To the created slurry, AA-TEMPO (0.05 g) and NaBr (1.25 g) were added. Due to the higher solubility of AA-TEMPO over the typical alternative TEMPO, a premixed solution was not required. The mixture was stirred for 30 minutes to create a homogenous mixture, and then cooled to 15° C.

NaOCl (13-14% in water, 40 g) was then fed into the reaction vessel over a time period of 3-6 hours. The slow addition of basified NaOCl_{(aq)} allowed the reaction pH to climb slower than the rate the reaction produces acid, therefore allowing the pH of the reaction to be controlled purely by the addition of NaOCl_{(aq)}, with a pH of around 10 being maintained. This also reduced the risk of chlorine gas being produced, which could otherwise be caused if hydrogen chloride was added instead.

Once the pH of the reaction was stable and the free chlorine was below 100 ppm (determined by use of over-the-counter chlorine test strips), the pH of the reaction was brought down to between 3 and 5 on addition of aqueous hydrochloric acid. Reducing the pH causes the oxidised cellulose particles to flocculate and loose viscosity. The excess salt and catalyst was washed out by filtration.

A higher solids material was made with less water needed for washing when filtration (linear or tangential) was used. If centrifugation was alternatively used a large excess of water would be required to remove the same amount of salt, and the resulting oxidised cellulose material would be of lower solid content.

Once the salts were removed, the product was neutralised with aqueous sodium hydroxide to between pH 6 and 8 (the amount of sodium hydroxide would be determined by the level of oxidation of the material) to provide neutralised oxidised cellulose.

The neutralised oxidised cellulose was then homogenised in a high pressure homogeniser (dispersed by Silverson Homogeniser at 8,600 rpm for 15 minutes) in order to form the dispersion using high shear.

The resulting dispersed oxidised cellulose had a D(v,0.5)p value of 54.597, a D(v,0.1)p value of 17.034, and a D(v,0.9)p value of 155.538.

### EXAMPLES 2 TO 5

### Preparing Oxidised Cellulose

Example 1 was repeated with variation to the temperature of the reaction and the oxidation level of the cellulose. All other reactants and conditions were identical as described for Example 1. The temperatures of reaction and level of oxidation for each of Examples 2 to 5 are shown in Table 1.

TABLE 1
Variation of temperature and oxidation levels

| | Temperature | Level of |
|---|---|---|
| Example No. | (° C.) | Oxidation (%) |
| 2 | 5 | 25 |
| 3 | 5 | 19 |
| 4 | 15 | 30 |
| 5 | 15 | 18.5 |

The particle sizes and particle size distribution values for Examples 2 to 5 are shown in Table 2.

TABLE 2
Particle sizes for dispersed oxidised cellulose

| Example No. | D(v, 0.5)ₚ | D(v, 0.1)ₚ | D(v, 0.9)ₚ |
|---|---|---|---|
| 2 | 60.107 | 16.803 | 177.012 |
| 3 | 53.788 | 14.492 | 178.621 |
| 4 | 60.547 | 21.497 | 154.288 |
| 5 | 54.419 | 14.551 | 183.43 |

### EXAMPLE 6

### Preparing Oxidised Cellulose

Example 6 was prepared in the same way with the same reactants as described for Example 1. The only difference was the use of alternative raw material cellulose particles having a D(v,0.5)s value of 59.373, a D(v,0.1)s value of 14.578, and a D(v,0.9)s value of 270.555.

The neutralised oxidised cellulose was then homogenised in a high pressure homogeniser (dispersed by Silverson Homogeniser at 8,600 rpm for 15 minutes) in order to form the dispersion using high shear. After dispersion, glycerin was added to the water phase, and the composition heated to 70° C. The oil phase was then combined and heated to 70° C. Phases A and B (as shown in Table 3) were combined with fast stirring and homogenised for 5 minutes. The homogenised composition was stirred and cooled to 35° C., and Phase C (as shown in Table 3) was then added, with ingredients pre-dissolved, with stirring. The pH was then adjusted to between 5.5 and 6.5.

The viscosity values detailed for dispersion Examples 1 to 6 were determined with reference to a 2 wt. % oxidised cellulose in the formulation as shown in Table 3. The formulation as detailed in Table 3 was selected to represent how the oxidised cellulose behaved in a possible end-use personal care formulation.

TABLE 3
Formulation for viscosity measurement for Examples 1 to 6

| | Amount Present/ | Amount Present |
|---|---|---|
| | wt. % | in 300 g Batch |
| Phase A | | |
| Polyglyceryl-3 methylglucose | 3.00 | 9.00 |
| distearate | | |
| Caprylic/capric triglyceride | 3.00 | 9.00 |
| Cetyl alcohol | 2.00 | 6.00 |
| Theobroma grandiflorum | 1.50 | 4.50 |
| C12-15 alkyl benzoate | 10.00 | 30.00 |
| Ethylhexyl methoxycinnamate | 3.00 | 9.00 |
| Phase B | | |
| Water | 60.83 | 182.49 |
| Glycerin | 3.00 | 9.00 |
| Oxidised cellulose | 2.00 | 6.00 |
| Phase C | | |
| Glucono-1,5-lactone & sodium | 1.00 | 3.00 |
| benzoate (75:25) | | |
| Water | 10.67 | 32.01 |

Zero-shear viscosity measurements were obtained for the oxidised cellulose dispersions of Examples 1 to 6, and these are shown in Table 4.

TABLE 4
Zero-shear viscosity values

| Example No. | Zero-Shear Viscosity (Pa · s) |
|---|---|
| 1 | 11500 |
| 2 | 15530 |
| 3 | 12670 |
| 4 | 14160 |
| 5 | 21270 |
| 6 | 20580 |

From these results it can be see that in the oxidation ranges 18-30% using 5-30° C. reactions gave acceptable zero-shear viscosities.

### Examples of Variation of Dispersion Pressure

Further samples of oxidised cellulose (at 28% oxidation) were prepared and washed via membrane filtration and diluted with water to 4 wt. % solids. This was neutralised to pH 6.5 using a 10% solution of sodium hydroxide.

The neutralised oxidised cellulose was then added to the high pressure homogeniser (GEA Niro PANDA 2 stage continuous flow homogeniser) with the second stage set to 150 bar (sample D1). 50 mL of sample was collected and the first stage was then turned to increase the pressure to 300 bar (sample D2) and another 50 mL was collected.

The pressure was increase to 600 bar (sample D3), 900 bar (sample D4), 1,200 bar (sample D5), 1,500 bar (sample D6), and 2,000 bar (sample D7) with 50 mL samples collected at each pressure interval. The created gels were allowed to rest for a minimum of 24 hours before testing. In each case, a aqueous dispersion of 4 wt. % oxidised cellulose was formed.

All rheology gathering experiments were performed on a Haake Rheostress 600 instrument at 25° C. with a 35 mm 2° titanium cone against a plate with a 104 nm gap. All samples were allowed to equilibrate at temperature for 120 seconds before measurements were taken. The viscosity measurements under shear were measured by slowly ramping the shear rate up to 500 revolutions per second (rps), then slowly ramping down the shear rate back to 0 rps. The yield point and zero shear viscosities were measured by increasing the shear stress from 0 to 150 Pa.

Table 5 shows the zero-shear viscosity and yield point results for samples D1 to D7, each being homogenised at different pressures as noted above.

TABLE 5
Zero-shear viscosity and yield points for different
homogenisation pressures

| Sample | Zero Shear Viscosity in Pa · s (η₀) | Yield point in Pa (τ₀) |
|---|---|---|
| D1 | 3,215 | 7.0 |
| D2 | 8,419 | 16.2 |
| D3 | 12,780 | 36.7 |
| D4 | 13,380 | 47.1 |
| D5 | 16,660 | 53.1 |
| D6 | 13,290 | 53.7 |
| D7 | 12,410 | 39.7 |

Yield point values represent the amount of shear required to break the gel. A low yield point suggests only a small amount of force is required, i.e. to get from zero-shear viscosity to the under shear viscosity. Table 5 clearly shows that by changing the pressure of homogenisation the behaviour of the resulting dispersion can be dramatically altered with increasing viscosity and yield point up to a maximum at 1,200 bar.

Shear viscosity values were also measured for samples D3 to D5. Table 6 shows the shear viscosities at 5 rps and 40 rps.

TABLE 6
Shear viscosities at various shear rates

Shear Viscosity
(η in Pa · s) at
given shear rate

| Sample | 5 rps | 40 rps |
|---|---|---|
| D3 | 22.52 | 3.986 |
| D4 | 20.92 | 3.089 |
| D5 | 28.92 | 3.416 |

The results in Table 6 show that under shear the oxidised cellulose loses most of its viscosity, i.e. that it is shear thinning.

Particle size values were also obtained for the dispersed oxidised cellulose samples D1 to D7. Table 7 shows the particle size values obtained.

TABLE 7
Particle size data of oxidised cellulose dispersed at
different pressures

| Sample | D(v, 0.1) μm | D(v, 0.5) μm | D(v, 0.9) μm |
|---|---|---|---|
| D1 | 15.145 | 39.097 | 101.810 |
| D2 | 12.166 | 30.604 | 69.365 |
| D3 | 9.843 | 24.249 | 56.185 |
| D4 | 8.792 | 21.596 | 50.613 |
| D5 | 8.424 | 21.035 | 50.352 |
| D6 | 7.980 | 19.861 | 47.675 |
| D7 | 7.491 | 17.35 | 38.950 |

The results in Table 7 shows that there is a decrease in observable particle size until 900 bar, whereafter only slight changes in size occur until 2,000 bar at which point another drop is observed.

It has therefore been shown that the oxidised cellulose dispersions can have rheology tuned not only by changing the cellulose particles raw material, but also by changing the shear pressure used to disperse the produced oxidised cellulose. The oxidised cellulose made generally has a high zero-shear viscosity which makes it a good suspending agent, and also has low viscosity when under shear.

### Elastic Modulus/Viscous Modulus Values

Elastic modulus and viscous modulus values were measured for sample D3 (homogenised at 600 bar). The values obtained are shown in Table 8.

TABLE 8
Elastic and viscous modulus values

| | Elastic/storage modulus | Viscous/loss modulus |
|---|---|---|
| Sample | (G′ average) | (G″ average) |
| D3 | 1359.9 | 193.8 |

The large difference in G′ and G″ values suggest that the formed dispersion would have good rheology modification properties.

### Skin Feel Results

Skin feel tests were performed using dispersed oxidised cellulose D2, D3, and D4 as described above. Tests were done by assessing the “skin feel” provided by each sample, with values given on a scale of very poor, poor, average, good, and very good. The test results are shown in Table 9.

TABLE 9
Elastic and viscous modulus values

| Sample | Skin Feel Result |
|---|---|
| D2 | Good |
| D3 | Very Good |
| D4 | Very Good |

The skin feel test results shown that use of dispersions of oxidised cellulose of the present invention provide for good results in end use personal care formulations.

It is to be understood that the invention is not to be limited to the details of the above embodiments, which are described by way of example only. Many variations are possible.

1. A method of preparing oxidised cellulose comprising;
(i) reacting cellulose particles, having a median volume particle diameter value (D(v,0.5)s) in the range from 30 μm to 70 μm, in the presence of at least one nitroxyl radical catalyst, at least one halide co-catalyst, and at least one oxidant, wherein the temperature of the reaction is in the range from 1° C. to 30° C., and the pH is in the range from 8 to 12, to produce intermediate oxidised cellulose particles;
(ii) neutralising said intermediate oxidised cellulose particles; and
(iii) applying shear at a pressure in the range from 200 to 1,500 bar to said neutralised oxidised cellulose particles to produce oxidised cellulose particles having a median volume particle diameter value (D(v,0.5)p) of less than D(v,0.5)s, and a ratio of D(v,0.5)s to D(v,0.5)p in the range from 1.1 to 5.0:1, said oxidised cellulose particles having an oxidation value in the range from 15% to 30%.

2. A method of preparing oxidised cellulose comprising;
(i) reacting cellulose particles, having a median volume particle diameter value (D(v,0.5)s) in the range from 30 μm to 70 μm, in the presence of at least one nitroxyl radical catalyst, at least one halide co-catalyst, and at least one oxidant, wherein the temperature of the reaction is in the range from 1° C. to 30° C., and the pH is in the range from 8 to 12;
(ii) to produce intermediate oxidised cellulose particles having a median volume particle diameter value (D(v,0.5)i) in the range from 35 μm to 80 μm, and a ratio of D(v,0.5)i to D(v,0.5)s in the range from 0.8 to 1.65:1;
(iii) neutralising said intermediate oxidised cellulose particles; and
(iv) applying shear at a pressure in the range from 200 to 1,500 bar to said neutralised oxidised cellulose particles to produce oxidised cellulose particles having a median volume particle diameter value (D(v,0.5)p) of less than D(v,0.5)s, and a ratio of D(v,0.5)s to D(v,0.5)p in the range from 1.1 to 5.0:1, said oxidised cellulose particles having an oxidation value in the range from 15% to 30%.

3. A method of preparing intermediate oxidised cellulose comprising;
(i) reacting cellulose particles, having a median volume particle diameter value (D(v,0.5)s) in the range from 30 μm to 70 μm, in the presence of at least one nitroxyl radical catalyst, at least one halide co-catalyst, and at least one oxidant, wherein the temperature of the reaction is in the range from 1° C. to 30° C., and the pH is in the range from 8 to 12;
(ii) to produce intermediate oxidised cellulose particles having an oxidation value in the range from 15% to 30%, a median volume particle diameter value (D(v,0.5)i) in the range from 35 μm to 80 μm, and a ratio of D(v,0.5)i to D(v,0.5)s in the range from 0.8 to 1.65:1.

4. The method according to claim 1, wherein the cellulose particles starting material have an α-cellulose content greater than 70 wt. %.

5. The method according to claim 1, wherein the cellulose particles starting material have a carboxyl content of less than 5 mol. %.

6. The method according to claim 1, wherein the cellulose particles starting material have a D(v,0.9)s value of less than 350 μm, and a D(v,0.1)s value of greater than 5 μm.

7. The method according to claim 1, wherein the cellulose particles starting material have a ratio of D(v,0.9)s to D(v,0.1)s values in the range from 30:1 to 6:1.

8. The method according to claim 1, wherein the nitroxyl radical catalyst is selected from: 2,2,6,6-tetramethyl-piperidin-1-yloxyl (TEMPO); 4-amino-2,2,6,6-tetramethylpiperidine-1-oxyl; 4-hydroxy-2,2,6,6-tetramethylpiperidine 1-oxyl; 4-oxo-2,2,6,6-tetramethyl-1-piperidinyloxy; 2,2,6,6-tetramethyl-1-piperidinyloxy; and 4-acetamido-2,2,6,6-tetramethylpiperidine 1-oxyl (AA-TEMPO).

9. The method according to claim 1, wherein the oxidant is selected from sodium hypochlorite, lithium hypochlorite, potassium hypochlorite, or calcium hypochlorite.

10. The method according to claim 1, wherein the proportion of intermediate oxidised cellulose in the form of aggregates compared to the proportion in the form of individual particles is in the range from 0.7 to 1.3:1.3 to 0.7.

11. The method according to claim 1, wherein the intermediate oxidised cellulose have a D(v,0.5)i value in the range from 45 μm to 75 μm.

12. The method according to claim 1, wherein the intermediate oxidised cellulose have a D(v,0.9)i value of less than 350 μm, and a D(v,0.1)i value of greater than 10 μm.

13. The method according to claim 1, wherein the oxidised cellulose has a D(v,0.5)p value of less than 40 μm.

14. The method according to claim 13, wherein the oxidised cellulose has a D(v,0.9)p value of less than 120 μm and a D(v,0.1)p value of greater than 5 μm.

15. The method according to claim 13, wherein the ratio of the values of D(v,0.5)s to D(v,0.5)p is in the range from 1.1 to 5:1.

16. The method according to claim 13, wherein the ratio of the values of D(v,0.5)i to D(v,0.5)p is in the range from 1.5 to 8:1.

17. The method according to claim 13, wherein the ratio of the values of D(v,0.9)s to D(v,0.9)p is in the range from 1.5 to 8:1.

18. The method according to claim 13, wherein the ratio of the values of D(v,0.9)i to D(v,0.9)p is in the range from 1.5 to 8:1.

19. The method according to claim 1, wherein the oxidised cellulose has a zero-shear viscosity in the range from 6,000 to 30,000 Pa·s.

20. The method according to claim 1, wherein oxidised cellulose has a yield point greater than 5 Pa.

21. The method according to claim 1, wherein the oxidised cellulose has a shear viscosity value in the range from 15 Pa·s to 35 Pa·s.

22. The method according to claim 1, wherein the oxidised cellulose has an elastic/storage modulus value (G′) in the range from 800 to 2,000 Pa.

23. The method according to claim 1, wherein the oxidised cellulose has a viscous/loss modulus value (G″) in the range from 50 to 300 Pa.

24. Oxidised cellulose particles obtainable by the methods of claim 1.

25. Intermediate oxidised cellulose particles obtainable by the method of claim 3.

26. Intermediate oxidised cellulose particles having an oxidation value in the range from 15% to 30%, and a median volume particle diameter value (D(v,0.5)i) in the range from 35 μm to 80 μm.

27. Oxidised cellulose particles having an oxidation value in the range from 15% to 30%, a median volume particle diameter value D(v,0.5)p of less than 40 μm, and an equivalent spherical diameter value corresponding to 90% of the volume of all the particles (D(v,0.9)p) of less than 120 μm.

28. A composition comprising in the range from 1 wt. % to 16 wt. % oxidised cellulose particles obtainable by the method of claim 1.

29. A composition comprising in the range from 1 wt. % to 16 wt. % oxidised cellulose particles having an oxidation value in the range from 15% to 30%, and a median volume particle diameter value (D(v,0.5)p) of less than 40 μm.
